SEQUENCE LISTING (24 sequences)

SEQ ID NO: 1 - DNA, 1458 bp, Flavobacterium sp.
  CDS (1)..(1455): DHHase
atgacccattacgatctcgtcattcgcggaggaaccgtcgccacggcgagtgattgcttccgggcggatgttgcggtcaccgacggcaggatcgtcgccatcggcgagaatctcgggcccgccgcccgcgagatctcggcggaaggccgcctcgttctgcccggcggcgtcgattcccattgccatatcgaggagccgcaggtcggcgaggtgcgcaacgcggaaaccttcgcctccgccacctcttcggcggccgcgggcggcacgacgacggtcatttccttctctcagcaggtgaaaggcggcggcatcaccgaggccctgcgcgactatcacgagaaggcgacccgcgcgctgatcgactattccttccatctcgtcgtgaccgatcccacggatgcggtgctggaggaactggtgccgctgatcgaggaagggcaccggtcgctgaagatcttcatgacctataccaatgtcgtgctcgacgacgagcagacgctgcgcgtgctggcgctcgcccgcaagaccggcgctctcgtcacggtgcatgccgagaaccatgccgcgatcatgtacctcacgcgtgccttggaaaaggccgggctcaccgccccgaagtaccacgcctgggccaagcccatgccggtggaacgggaggcctgccaccgcatcatcatgctctccgaactgctggatgttcccattcagatcttccacgtttccggggcggaggcggccgaggaaatccgccgcgcgcagaatcgcggcctcaaggtcttcggcgagacctgcccgcaatatctgatgctgacggcggcggatctcgaccggccggatttcgagggtgcgaaattcctgtgcagccccgcgccgcgcacgacggccgatcaggaagccctgtgggactatatccgcacgggaacgatcggggtcgtgtcgtccgaccacgcgccgaaccgcttcgacgatccgcacggcaagaaggtcggcggggtggacgcgcccttcagcgccattccgaacggcgtgccggggctggcgacgcgcctgccgatcctgttttccgaaggggtcgtcaaggggcgcatcgacctcaacaccttcgtcgcgctgacggcggccaatccggcgaagctctttggcctgcatcccagaaagggcaccatcgccgtcggagccgatgcggatatcgcgatctgggatcccgagcgcaaggtgacgatccgcaacgacatgctgcatcacggcgtcgactacacggtctacgagggcgtcgaggtgaccggatggccggtgatgacgctctcgcgcggcgaggtggtctatgatgacggcgcggtggtcggcaagccgggtcacgggcgcttccttgcccgcggtccctatgacgcgatcaagccgcgcggcaggcatgtgacgcctttcgacccggtgacgggcgaagtggaaacggcctga

SEQ ID NO: 2 - protein, 485 aa, Flavobacterium sp.
MTHYDLVIRGGTVATASDCFRADVAVTDGRIVAIGENLGPAAREISAEGRLVLPGGVDSHCHIEEPQVGEVRNAETFASATSSAAAGGTTTVISFSQQVKGGGITEALRDYHEKATRALIDYSFHLVVTDPTDAVLEELVPLIEEGHRSLKIFMTYTNVVLDDEQTLRVLALARKTGALVTVHAENHAAIMYLTRALEKAGLTAPKYHAWAKPMPVEREACHRIIMLSELLDVPIQIFHVSGAEAAEEIRRAQNRGLKVFGETCPQYLMLTAADLDRPDFEGAKFLCSPAPRTTADQEALWDYIRTGTIGVVSSDHAPNRFDDPHGKKVGGVDAPFSAIPNGVPGLATRLPILFSEGVVKGRIDLNTFVALTAANPAKLFGLHPRKGTIAVGADADIAIWDPERKVTIRNDMLHHGVDYTVYEGVEVTGWPVMTLSRGEVVYDDGAVVGKPGHGRFLARGPYDAIKPRGRHVTPFDPVTGEVETA

SEQ ID NO: 3 - DNA, 915 bp, Flavobacterium sp.
  CDS (1)..(912): DCHase
atgccaggaaagatcattctcgcggtgggccagcttggacccatcgcccgcagcgacagccgcgaacaggtggtgaagcggctcgtcgacatgctggaggaggccgcggcccgcaattcggatttcatcgtctttcccgagctggcgctgaccaccttcttcccccgctggtatttcaccgacgaggcggaactggacgcgttctacgagacggaaatgccgagcccggtgacacgcccgctgttcgaggccgccgcgaaacacggcgtcgggttcaatctcggcttcgcggagctcgtgtcggagggcggcaggaagcgccgcttcaacacgtccatcgccgtcgacaggaccggcaggatcatcggcaagtaccgcaagatccatctgccgggccacaaggactatgaagactaccgcccgttccagcatctggagaagcggtacttcgagaccggcaatctcggattcccggtctatgaggtcgatggtttcaagatgggcatgttcatctgcaacgaccggcgctggccggagacctggcgcgtgatgggtctcaagggcgcggagctgatctgcggcggctacaacacgccgctgcacaatccgccggtgccgcagcacgaccagctcagctccttccaccatctcctgtcgatgcaggcgggcgcctaccagaacggtgcgtggaccgccggagcgggcaaggtcggggtggaggaaggctgcatgctgctcggccattcctgcatcgtcgcgcccaccggggagcttgccgcgctcaccaacacgctggaggacgaggtcatcacggccgctgccgattttgcccgctgccgggaaatccgcgagaacatcttcaatttcgagcagcatcgcgagccgcaggaatacgggttgatcgccgccgtctga

SEQ ID NO: 4 - protein, 304 aa, Flavobacterium sp.
MPGKIILAVGQLGPIARSDSREQVVKRLVDMLEEAAARNSDFIVFPELALTTFFPRWYFTDEAELDAFYETEMPSPVTRPLFEAAAKHGVGFNLGFAELVSEGGRKRRFNTSIAVDRTGRIIGKYRKIHLPGHKDYEDYRPFQHLEKRYFETGNLGFPVYEVDGFKMGMFICNDRRWPETWRVMGLKGAELICGGYNTPLHNPPVPQHDQLSSFHHLLSMQAGAYQNGAWTAGAGKVGVEEGCMLLGHSCIVAPTGELAALTNTLEDEVITAAADFARCREIRENIFNFEQHREPQEYGLIAAV

SEQ ID NO: 5 - DNA, 711 bp, Microbacterium liquefaciens
  CDS (1)..(708): HRase
atgagaatccatgtcatcaatcccaacagctcggtggatctcaccgatgcggtggccgaggcggcgcgaagtgtggtgtcaccgggaaccaccatcaccgcggtcaacccttcgaagggccccacggtcatcgagggcagttacgacgaggtgctggccacgtatcacctcgtcgaagaggtccgccgcgcggagcgcgaagaccgaccggacgcctacgtcatcgcctgtttcggcgatccaggtctcgacgccgtcagggagctcaccgacaggcccgtggtcggaatcgccgaggcggcgatccagatgacgagcttcgtcgccgcgagcttctccatcgtgagcatcctcccgcgcgtgcgcaagcatctgcacgagctggtgcaccgggcgggggcaacggatcgactcgcctcactcaagcttccggatctcggagtgctcgcattccacgaggacgaggcagcggcgttcgagaccctccggcgcgtggcaggtgaggcggtgcgcgaggacggcgcggagtcgatcgtgctcggctgcgcgggcatggccggattcgccagacagctgagtgaagagctcggcgtccccgtcatcgacgcggtcgaggcagcctgccgcgtcgcggagagcctcgtcgccctggggtaccgcaccagcaaggcgaacacctaccaagcacccaccgagaagcagtacctctaa

SEQ ID NO: 6 - protein, 236 aa, Microbacterium liquefaciens
MRIHVINPNSSVDLTDAVAEAARSVVSPGTTITAVNPSKGPTVIEGSYDEVLATYHLVEEVRRAEREDRPDAYVIACFGDPGLDAVRELTDRPVVGIAEAAIQMTSFVAASFSIVSILPRVRKHLHELVHRAGATDRLASLKLPDLGVLAFHEDEAAAFETLRRVAGEAVREDGAESIVLGCAGMAGFARQLSEELGVPVIDAVEAACRVAESLVALGYRTSKANTYQAPTEKQYL

SEQ ID NO: 7 - DNA, 40 bp, Artificial Sequence
  primer (7)
gtatcacgaggccctagctgtggtgtcatggtcggtgatc

SEQ ID NO: 8 - DNA, 40 bp, Artificial Sequence
  primer (8)
ttcggggattccatatgataccctttttacgtgaacttgc

SEQ ID NO: 9 - DNA, 38 bp, Artificial Sequence
  primer (9)
ggggggggcatatgcgacctccttattacgtgaacttg

SEQ ID NO: 10 - DNA, 67 bp, Artificial Sequence
  primer (10)
aattctgttgacaataattcatcgaactagttaactagtacgcaagttcacgtaaaaagggtggtac

SEQ ID NO: 11 - DNA, 26 bp, Artificial Sequence
  primer (11)
ggggtaccatgacccattacgatctc

SEQ ID NO: 12 - DNA, 26 bp, Artificial Sequence
  primer (12)
gctctagacgtcctgtcctttccgcc

SEQ ID NO: 13 - DNA, 26 bp, Artificial Sequence
  primer (13)
ggggtaccggagaggaatatgccagg

SEQ ID NO: 14 - DNA, 26 bp, Artificial Sequence
  primer (14)
gctctagagccgcgccgctcagacgg

SEQ ID NO: 15 - DNA, 25 bp, Artificial Sequence
  primer (15)
cgtctagatgttgacaattaatcat

SEQ ID NO: 16 - DNA, 32 bp, Artificial Sequence
  primer (16)
cgctgcagtcaggccgtttccacttcgcccgt

SEQ ID NO: 17 - DNA, 34 bp, Artificial Sequence
  primer (17)
ggaattccatatgccaggaaagatcattctcgcg

SEQ ID NO: 18 - DNA, 29 bp, Artificial Sequence
  primer (18)
cggaattctcaggccgtttccacttcgcc

SEQ ID NO: 19 - DNA, 36 bp, Artificial Sequence
  primer (19)
gggaattccatatgacccattacgatctcgtcattc

SEQ ID NO: 20 - DNA, 29 bp, Artificial Sequence
  primer (20)
cggaattctcaggccgtttccacttcgcc

SEQ ID NO: 21 - DNA, 30 bp, Artificial Sequence
  primer (21)
caagttcacgtaataaggaggtcgcatatg

SEQ ID NO: 22 - DNA, 29 bp, Artificial Sequence
  primer (22)
cgcggatccttagaggtactgcttctcgg

SEQ ID NO: 23 - DNA, 32 bp, Artificial Sequence
  primer (23)
cgggaattccatatgcgtatccatgtcatcaa

SEQ ID NO: 24 - DNA, 29 bp, Artificial Sequence
  primer (24)
cgcggatccttagaggtactgcttctcgg

(19) United States
(12) Patent Application Publication
(10) Pub. No.: US 2005/0214912 A1
(43) Pub. Date: Sep. 29, 2005

(54) Method for producing an optically active amino acid

(76) Inventors: Hiroyuki Nozaki, Kawasaki-shi (JP); Kunihiko Watanabe, Kawasaki-shi (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/091,892

(22) Filed: Mar. 29, 2005

(30) Foreign Application Priority Data
Mar. 29, 2004 (JP) 2004-095983

(51) Int. Cl. C12P 13/04; C12N 9/90; C12N 1/21

(52) U.S. Cl. 435/106; 435/252.33; 435/233

(57) ABSTRACT

A method for producing an optically active amino acid comprising contacting a 5-substituted hydantoin with a group of enzymes including both a hydantoinase and a carbamoylase, wherein the method is carried out in an aqueous solution with a dissolved oxygen concentration of 1.5 ppm or less. Optically active amino acids such as D-tyrosine may be produced with high efficiency.

### CROSS-REFERENCE TO RELATED APPLICATIONS

The present application claims the benefit of priority under 35 U.S.C. §119 based on the Japanese Patent Application No. 2004-095983 filed on Mar. 29, 2004, the entire disclosure of which is incorporated herein by reference.

### BACKGROUND OF THE INVENTION

1. Field of the Invention

The present invention relates to a method for producing an optically active amino acid using enzymes such as those derived from a microorganism.

2. Brief Description of the Related Art

An optically active amino acid is useful as a raw material and as a synthetic intermediate of foods and pharmaceuticals.

One of the known methods for producing the optically active amino acid includes using a microorganism or an enzyme. For example, JP-P-S54-2274 B, JP-P-S54-8749 B and JP-P-S60-214889 A disclose methods for producing an L-amino acid using a 5-substituted hydantoin as a substrate. JP-P-S62-205790 A, JP-P-H10-80297 A, JP-P-H10-286098 A, JP-P-S61-257931 A and Applied and Environmental Microbiology, vol. 54, No. 4, p. 984-989 disclose a variety of methods for producing a D-amino acid using a microorganism or an enzyme.

However, these methods give an extremely low concentration of D-tyrosine accumulation, and a low optical purity of D-tyrosine. Therefore, these methods are unsatisfactory for industrial production of D-tyrosine.

### SUMMARY OF THE INVENTION

It is an object of the present invention to provide a method for industrially advantageous production of an optically active amino acid with high efficiency and high yield.

According to the present invention, there is provided a method for producing an optically active amino acid comprising contacting a 5-substituted hydantoin with a group of enzymes including both a hydantoinase and a carbamoylase, wherein the step is carried out in an aqueous solution with a dissolved oxygen concentration of 1.5 ppm or less.

The group of enzymes may further comprise hydantoin racemase. The hydantoinase and carbamoylase may be produced from cells having a hydantoinase gene and a carbamoylase gene. The hydantoin racemase may be produced from cells having a hydantoin racemase gene. The step may be carried out in a state in which the aqueous solution is placed under an atmosphere comprising an inert gas. The cells may be Escherichia coli. The optically active amino acid may be tyrosine. The tyrosine may be D-tyrosine.

According to the method for producing the optically active amino acid of the present invention, the yield of the desired optically active amino acid may be remarkably increased.

The other objects, features and advantages of the present invention are specifically set forth in or will become apparent from the following detailed descriptions of the invention when read in conjunction with the accompanying drawings.

### BRIEF DESCRIPTION OF THE DRAWINGS

FIG. 1 is a schematic view showing construction of the plasmid pTrp4R in Example 1.

FIG. 2 is a schematic view showing construction of the plasmid pTrp8HR in Example 1.

FIG. 3A is a graph showing the relationship of reaction time and conversion yield in the production of N-carbamoyl-D-tyrosine in Example 1.

FIG. 3B is a graph showing the relationship of reaction time and oxidation-reduction potential (ORP) in the production of N-carbamoyl-D-tyrosine in Example 1.

FIG. 4 is a view showing the trp promoter cassette used in Example 2.

FIG. 5 is a schematic view showing the construction of the plasmid pTrpHrCr in Example 2.

FIG. 6 is a schematic view showing the construction of the plasmid pTrp8CH in Example 2.

FIG. 7A is a graph showing the relationship of reaction time and conversion yield in the production of D-tyrosine in Example 2 and Comparative Example 1.

FIG. 7B is a graph showing the relationship of reaction time and oxidation-reduction potential (ORP) in the production of D-tyrosine in Example 2 and Comparative Example 1.

### DETAILED DESCRIPTION OF THE PREFERRED EMBODIMENTS

As a result of extensive study in the light of the above object, the present inventors have found that generation of a colored insoluble product is suppressed and a remarkably high yield can be realized in the conversion of 5-substituted hydantoin to an optically active amino acid using a group of enzymes including both a hydantoinase and a carbamoylase by performing the reaction in an aqueous solution under a state of low oxygen concentration, and thereby completed the present invention.

In the method for producing the optically active amino acid of the present invention, a 5-substituted hydantoin is used as a substrate. The 5-substituted hydantoin is a hydantoin derivative having a substituent at position 5. A desired amino acid may be obtained by appropriately selecting this substituent at position 5. Specifically, tyrosine may be produced by the use of 5-(4-hydroxybenzyl)hydantoin as the substrate, tryptophan may be produced by the use of 5-((3-indolyl)methyl)hydantoin as the substrate, and o-benzylserine may be produced by the use of 5-(phenoxymethyl)hydantoin as the substrate. However, the amino acids of the present invention are not limited thereto. Any amino acid may be produced by the use of a hydantoin derivative having a corresponding substituent at position 5. As the 5-substituted hydantoin, any D-isomer, any L-isomer, and a mixture thereof (DL isomers) may be used.

In the production method of the present invention, the 5-substituted hydantoin is used as the substrate and a reaction is performed with a specific group of enzymes. The group of the enzymes for the reaction includes a hydantoinase and a carbamoylase. It is particularly preferable that the group of the enzymes for the reaction further include a hydantoin racemase, in terms of an effective use of the opposite enantiomer of the 5-substituted hydantoin isomer for the desired optical isomer of the amino acid (i.e., D-5-substituted hydantoin when producing an L-amino acid whereas L-5-substituted hydantoin when producing a D-amino acid), even when such an opposite enantiomer is present in the substrate for the reaction. The manner of conducting the reaction with such a plurality of enzymes is not particularly limited as long as the aforementioned specific enzymes are involved in the reaction, and the reaction may be performed in a system in which the desired two or three enzymes are present in one reaction mixture to continuously perform the reaction. Alternatively, the reaction may be performed in a system in which the desired two or three enzymes are present in different reaction mixtures to separately perform enzymatic reactions.

When a D-amino acid is produced in the production method of the present invention, an example of the reaction may include the following reaction steps (I) to (III):

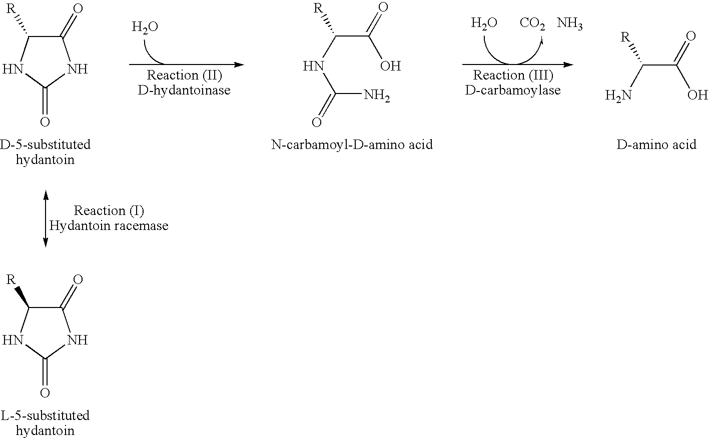

In the above reaction formula, R is an arbitrary substituent, and preferably represents a side chain of a known amino acid (e.g., the side chain of tyrosine is a 4-hydroxybenzyl group, the side chain of tryptophan is a 3-indolylmethyl group, and the side chain of o-benzylserine is a phenoxymethyl group).

The reaction steps in the present invention may be such that the reaction (II) and the reaction (III) are performed in one reaction mixture, or in separate reaction mixtures. The reaction (I) is a preferable aspect of the production method of the present invention.

Upon producing an L-amino acid, the reaction may be performed with an L-hydantoinase and an L-carbamoylase in the place of the D-hydantoinase and the D-carbamoylase in the aforementioned reaction.

As used herein, the D-hydantoinase refers to an enzyme which catalyzes the reaction to convert a D-5-substituted hydantoin into an N-carbamoyl-D-amino acid. The D-carbamoylase refers to an enzyme which catalyzes the reaction to convert the N-carbamoyl-D-amino acid into the D-amino acid. In the production of the D-amino acid, preferably, at least one of these two enzymes of the hydantoinase and the carbamoylase selectively catalyzes only the reaction utilizing the D-isomer as the substrate but substantially does not catalyze the reaction utilizing the L-isomer as the substrate. More preferably, both enzymes selectively catalyze only the reaction utilizing the D-isomer as the substrate.

The L-hydantoinase refers to an enzyme which catalyzes the reaction to convert an L-5-substituted hydantoin into an N-carbamoyl-L-amino acid. The L-carbamoylase refers to an enzyme which catalyzes the reaction to convert the N-carbamoyl-L-amino acid into the D-amino acid. In the production of the L-amino acid, preferably, at least one of these two enzymes of the hydantoinase and the carbamoylase selectively catalyzes only the reaction utilizing the L-isomer as the substrate but substantially does not catalyze the reaction utilizing the D-isomer as the substrate. More preferably, both enzymes selectively catalyze only the reaction utilizing the L-isomer as the substrate.

The hydantoin racemase refers to an enzyme which catalyzes the reaction to racemize hydantoin and/or any of the derivatives thereof.

Examples of the aforementioned enzymes may include those produced from cells having a gene which expresses any one of the corresponding enzymes. That is, it is possible to employ the enzymes produced by cells having a hydantoin racemase gene, a hydantoinase gene and a carbamoylase gene. For example, the three enzymes may be simultaneously produced using one strain of cells containing all three genes. Alternatively, the enzymes may be obtained by appropriately combining a plurality of strains each containing one or two of these three genes. More specifically, the enzymes may be obtained from cells containing both the hydantoin racemase gene and the hydantoinase gene and other cells containing the carbamoylase gene, which may be combined for use. The hydantoin racemase gene, the hydantoinase gene, the carbamoylase gene, cells having these genes, and the production of the enzymes using these cells will be separately described in detail later.

Specifically, the above reaction may be performed by culturing the cells in a medium to obtain a culture containing the desired enzymes and mixing the culture with the substrate. Alternatively, the reaction may also be performed by mixing the substrate with any of the microbial cells separated from the culture, the washed microbial cells, a treated product obtained by disrupting or lysing the cells, a crude enzyme solution obtained by collecting the enzyme therefrom, or a purified enzyme solution. Furthermore, it is also possible to perform the reaction to produce the optically active amino acid simultaneously with the cultivation of the aforementioned cells. In this case, the reaction mixture may contain nutritional elements necessary for the growth of the cells, such as carbon sources, nitrogen sources and inorganic ions, as well as organic trace nutritional elements such as vitamin and amino acids. It is not necessary to add the full amount of the substrate into the reaction mixture at the onset of the reaction, and the amount of substrate added may be divided and added separately.

In the production method of the present invention, the step of producing the optically active amino acid in accordance with the above reaction is performed in an aqueous solution with a dissolved oxygen concentration of 1.5 ppm or less, preferably 0.3 ppm or less. The amount of the dissolved oxygen in the aqueous solution may specifically be measured by an oxygen electrode meter (S-1 type, supplied from Biott Co., Ltd.) and an oxidation-reduction electrometer (supplied from Broadly James). A relative oxygen concentration may also be measured by the oxidation-reduction electrometer (supplied from Broadly James). The reaction at such a low dissolved oxygen concentration may be accomplished by placing the aqueous solution under an atmosphere replaced with an inert gas.

More specifically, the reaction at a low dissolved oxygen concentration may be performed under an inert gas atmosphere and bubbling the solution with the inert gas. Examples of the inert gas include nitrogen and argon. When the inert gas flow is introduced into a sealed reaction vessel, the gas discharged from an outlet may be analyzed by an oxygen gas analysis apparatus (type DEX-1562-2, supplied from Biott Co., Ltd.).

An oxidation-reduction potential (ORP) in the aqueous solution may be monitored to confirm that the reaction is maintained in a state of low oxygen concentration.

Generally, the dissolved oxygen concentration in an aqueous solution varies depending on temperature, type and amount of the dissolved substance. In the case of purified water at 37° C., a dissolved oxygen concentration in a state in which sufficient stirring and ventilation are performed, i.e., a saturated dissolved oxygen concentration is about 6.86 ppm (according to data in Seibutsukogaku Jikkensho (textbook for bioengineering experiments) edited by the Society for Biotechnology, Japan and issued by Baifukan, Japan). In the production method of the present invention, the reaction is performed by controlling the dissolved oxygen concentration to as low as 1.5 ppm or less, preferably 0.3 ppm or less. By performing the reaction at such a low dissolved oxygen concentration, a decrease in the yield of the optically active amino acid due to generation of an insoluble substance in the reaction system may be prevented. When the cells or a cell lysate is used as an enzyme source for the reaction, an enzyme which catalyzes an undesirable side reaction using oxygen in the reaction system as a substrate may contaminate the reaction system. However, even in such a case, the reaction at such a state of low oxygen concentration results in suppression of the undesired side reaction, which leads to good performance of the reaction. Specifically, the method for producing the optically active amino acid of the present invention may be favorably performed even in coexistence with an L-amino acid oxidase (EC 1.4.3.2) and a D-amino acid oxidase (EC 1.4.3.3).

In order to prevent the oxidation of the reaction substrate and the reaction product in the aqueous solution in which the reaction is performed, an antioxidant or a reducing agent may be added thereto. Examples of such an antioxidant and a reducing agent may include dithiothreitol and sodium sulfite. In the reaction, the amount of the antioxidant or the reducing agent to be added may be appropriately adjusted so that it is within the range where the objective reaction is not inhibited, and those skilled in the art may determine an adequate concentration by a preliminary experiment.

Although conditions for the above reaction are not particularly limited as long as the dissolved oxygen amount is in the aforementioned specific range, the reaction may specifically be performed by adjusting the temperature in an appropriate range of 25 to 40° C., and maintaining the pH in a range of 5 to 9. The reaction system may be left to stand, or stirred for a period of time in a range of 8 hours to 5 days. Maintaining the pH of the reaction system may be performed by appropriately adding an acid or an alkali, e.g., H₂SO₄ and NaOH while monitoring the pH value.

Quantification of the optically active amino acid synthesized in the reaction process may be rapidly performed using a well-known method. That is, a simple thin layer chromatography with, e.g., TPTLC or CHIR supplied from Merck may be utilized. When a higher analysis accuracy is required, a high performance liquid chromatography (HPLC) with an optical resolution column such as CHIRALPAK WH supplied from Daicel Chemical Industries, Ltd. may be used.

The synthesized optically active amino acid may be isolated and purified by known techniques. Such an isolation and purification may be performed by, e.g., contacting the reaction mixture after the completion of the reaction with an ion exchange resin which absorbs the optically active amino acid, followed by elution and crystallization of the amino acid. The eluent may also be decolorized by filtration through an active charcoal before crystallization.

The optically active amino acid to be produced by the production method of the present invention is not particularly limited, and may include tyrosine, tryptophan and o-benzylserine. In the method of the present invention, any D-isomer and any L-isomer amino acid may be produced by appropriately selecting the hydantoinase and the carbamoylase as described above.

Subsequently, the hydantoin racemase gene, the hydantoinase gene, the carbamoylase gene, and cells having them for use in the present invention, as well as the production of the enzymes using the cells will be described in detail hereinbelow.

As the hydantoin racemase gene, the hydantoinase gene and the carbamoylase gene, those already known may be used. For example, as the D-hydantoinase gene, the D-carbamoylase gene and the hydantoin racemase gene, genes encoded by DNAs of the following (A) to (C) may be used, respectively.

(A) A DNA Encoding the D-Hydantoinase Gene

The DNA may be selected from the following (i) to (iv):
(i) a DNA having a nucleotide sequence described in SEQ ID NO: 1;
(ii) a DNA having a nucleotide sequence which hybridizes with a DNA composed of a nucleotide sequence complementary to the nucleotide sequence described in SEQ ID NO:1 under stringent conditions;
(iii) a DNA encoding an amino acid sequence described in SEQ ID NO:2; and
(iv) a DNA encoding an amino acid sequence having substitution, deletion, insertion, addition and/or inversion of one or several amino acid residues in the amino acid sequence described in SEQ ID NO:2.
   (B) A DNA Encoding the D-Carbamoylase Gene

The DNA selected from the following (v) to (viii):
(v) a DNA having a nucleotide sequence described in SEQ ID NO:3;
(vi) a DNA having a nucleotide sequence which hybridizes with a DNA composed of a nucleotide sequence complementary to the nucleotide sequence described in SEQ ID NO:3 under stringent conditions;
(vii) a DNA encoding an amino acid sequence described in SEQ ID NO:4; and
(viii) a DNA encoding an amino acid sequence having substitution, deletion, insertion, addition and/or inversion of one or several amino acid residues in the amino acid sequence described in SEQ ID NO:4.
   (C) A DNA Encoding the Hydantoin Racemase Gene

The DNA selected from the following (ix) to (xii):
(ix) a DNA having a nucleotide sequence described in SEQ ID NO:5;
(x) a DNA having a nucleotide sequence which hybridizes with a DNA composed of a nucleotide sequence complementary to the nucleotide sequence described in SEQ ID NO:5 under stringent conditions;
(xi) a DNA encoding an amino acid sequence described in SEQ ID NO:6; and
(xii) a DNA encoding an amino acid sequence having substitution, deletion, insertion, addition and/or inversion of one or several amino acid residues in the amino acid sequence described in SEQ ID NO:6.

As used herein, “stringent conditions” refers to those under which a so-called specific hybrid is formed and no non-specific hybrid is formed. Specifically, examples thereof may include those whereby a pair of DNAs having high homology, e.g., DNAs having the homology of not less than 50%, more preferably not less than 80%, and still more preferably not less than 90% are hybridized to each other whereas DNAs having homology lower than the above are not hybridized to each other. Another example of stringent conditions includes washing according to an ordinary Southern hybridization, i.e., hybridization at a salt concentration equivalent to 1×SSC and 0.1% SDS at 60° C., preferably 0.1×SSC and 0.1% SDS at 60° C., and more preferably 0.1×SSC and 0.1 SDS at 65° C.

As used herein, “several” amino acid residues are within the range where a three-dimensional structure of the protein with the amino acid residues and a hydantoinase activity or a carbamoylase activity are not significantly impaired, and specifically from 2 to 50, preferably 2 to 30, and more preferably 2 to 10 amino acid residues.

The DNA of SEQ ID NOS 1 and 3 have been isolated and purified from the chromosomal DNA of Flavobacterium sp. AJ 11199 (FERM-P4229) strain (JP-P-S56-025119 B). Flavobacterium sp. AJ 11199 (FERM-P4229) strain is a microorganism originally deposited as Alcaligenes aquamarinus to Ministry of International Trade and Industry, Agency of Industrial Science and Technology, National Institute of Bioscience and Human-Technology on Sep. 29, 1977, but as a result of reidentification, it was found to be classified into Flavobacterium sp. Thus this microorganism has been deposited as Flavobacterium sp. AJ 11199 (domestic accession number: FERM-P4229) strain to International Patent Organism Depositary, National Institute of Advanced Industrial Science and Technology (Central No. 6, 1-1-1 Higashi, Tsukuba-shi, Ibaraki Prefecture, Japan), and further has been deposited under the international accession number FERM BP-8063 to the same facility on May 30, 2002 in accordance with the Budapest Treaty (original date of deposit under the Budapest Treaty: May 1, 1981).

The DNA of SEQ ID NO:5 has been isolated and purified from Microbacterium liquefaciens AJ3912 strain. Microbacterium liquefaciens AJ3912 strain was originally deposited as Flavobacterium sp. AJ3912 (FERM-P3133) to Ministry of International Trade and Industry, Agency of Industrial Science and Technology, National Institute of Bioscience and Human-Technology on Jun. 27, 1975, but as a result of reidentification, it was found to be classified into Aureobacterium liquefaciens. Further, due to a species name change, Aureobacterium liquefaciens has been classified into Microbacterium liquefaciens. Thus this microorganism has been deposited as Microbacterium liquefaciens AJ3912 strain (domestic accession number: FERM-P3133) to International Patent Organism Depositary, National Institute of Advanced Industrial Science and Technology, and further has been deposited under the international accession number FERM BP-7643 to the same facility on Jun. 27, 2001 in accordance with the Budapest Treaty (original date of deposit under the Budapest Treaty: May 1, 1981).

A plasmid for transformation may be constructed by ligating each DNA of the aforementioned (A) to (C) to, if necessary, other sequences such as the sequences of a promoter and a terminator at an upstream and a downstream thereof, and further ligating the resulting sequence to the other sequence. Each enzyme may be expressed by introducing this plasmid into a cell.

As the promoter, a rhamnose promoter and a trp promoter may be used, and in particular the trp promoter may be preferably used. The trp promoter is a promoter present upstream of a gene group encoding several enzymes involved in tryptophan biosynthesis, and is present in, e.g., Escherichia coli. In the present invention, the known trp promoter may be used. The trp promoter is commercially available in a form of a vector for cloning containing the trp promoter, or in a form of cells containing such a vector. By the use of the trp promoter, sufficient amounts of the hydantoinase and the carbamoylase may be expressed. The trp promoter does not require addition of an inducer unlike the rhamnose promoter which requires addition of rhamnose, whereby some steps and treatments for adding the inducer can be omitted in the production process of the amino acid. Furthermore, the trp promoter is not inhibited by glucose. Thus, even if glucose is used for culturing the transformed microorganisms at a high density in the amino acid production on an industrial scale, the expression of the enzyme is not suppressed thereby.

Examples of the terminator may include an rmB terminator, a T7 terminator, an fd phage terminator, a T4 terminator, and terminators of a tetracycline resistant gene and an Escherichia coli trpA gene. The rmB terminator is preferable in terms of making the plasmid more stable.

For selecting a transformant, it is preferable that the plasmid has a marker such as an ampicillin resistant gene and a kanamycin resistant gene.

A plasmid for the transformation may be constructed by ligating the DNA of the above (A) to (C), and other optional sequences such as a promoter, a terminator and a marker to a known vector. Specific examples of such vectors may include plasmids of a pHSG type (supplied from Takara Shuzo Co., Ltd.), a pUC type (supplied from Takara Shuzo Co., Ltd.), a pPROK type (supplied from Clontech), a pSTV type (supplied from Takara Shuzo Co., Ltd.), a pTWV type (supplied from Takara Shuzo Co., Ltd.), a pKK233-2 type (supplied from Clontech) and a pBR322 type, and derivatives thereof. As used herein, the “derivative” means a plasmid modified by substitution, deletion, insertion, addition or inversion of a nucleotide(s). As used herein, the “modification” may include a modification caused by mutagenesis with a mutagen or UV irradiation and a spontaneous mutation. Copy numbers of the plasmid and the derivatives thereof in a host cell may vary depending on the type of replication origin. High-copy plasmids may include plasmids of the pHSG type, the pUC type and the pPROK type, and low-copy plasmids may include plasmids of the pSTV type, the pTWV type, the pKK233 type and the pBR322 type.

A transformant may be obtained by introducing the plasmid for transformation obtained as above into a host cell. When a protein is produced on a large scale using a recombinant DNA technology, the host cell to be transformed may include a bacterial cell, an actinomycetal cell, a yeast cell, a fungal cell, a plant cell, and an animal cell. Since there are numerous findings for the technology of producing the protein on a large scale using enteric bacteria, the enteric bacteria, preferably Escherichia coli may be used. In particular, Escherichia coli JM1 09 strain, particularly (DE3) strain is preferable.

It is less practical to carry all three DNA of the above (A) to (C) on one plasmid due to the size of the enzyme gene. Therefore, it is preferable to construct plasmids each having one or two DNA of (A) to (C) and introduce a combination thereof as needed into the host to yield transformed cells having the desired genes. When a plurality of plasmids are constructed, all of the plasmids may be introduced into one strain of a host cell to yield one type of a transformant, and the resulting transformant may be cultured alone to collectively yield the desired enzymes. Alternatively, the plasmids may be introduced into different host cells to yield a plurality of transformants, and the transformants may be cultured to yield the desired enzymes each derived therefrom. The yielded enzymes may then be combined to use. For example, the desired enzymes may be obtained by constructing a plasmid carrying two of the DNAs (A) to (C) and another plasmid carrying the remaining one DNA, introducing these two plasmids into one strain of a host cell to yield one type of a transformant, and culturing the same to obtain the desired enzyme. Alternatively, the desired enzymes may be obtained by introducing these two plasmids into different host cells to yield two types of transformants, and co-culturing them or separately culturing them.

Manipulations for treating the plasmids, DNA fragments and enzymes, as well as the production and the selection of the transformants may be performed in accordance with known techniques such as those described in Molecular Cloning, A Laboratory Manual, 2nd Edition edited by J. Sambrook et al., 1989 (Cold Spring Harbor Laboratory Press).

As the cells having the hydantoin racemase gene, the hydantoinase gene and the carbamoylase gene, bacterial strains known publicly which produce the hydantoin racemase, the hydantoinase and/or the carbamoylase may also be used in addition to the aforementioned transformants.

The publicly known bacterial strains which produce the hydantoinase may specifically include a strain belonging to a genus Bacillus which produces a heat resistant enzyme. For example, the D-hydantoinase may be obtained from Bacillus stearothermophilus ATCC 31195 strain. The ATCC 31195 strain is available from American Type Culture Collection (address: 12301 Parklawn Drive, Rockville, Md., 20852, USA). It has been known that the L-hydantoinase is present in Bacillus sp. AJ 12299 strain (JP-P-S63-24894 A). Bacillus sp. AJ 12299 strain was deposited to Ministry of International Trade and Industry, Agency of Industrial Science and Technology, National Institute of Bioscience and Human-Technology on Jul. 5, 1986, and the accession number FERM-P8837 was allotted thereto. Then, the strain was transferred to International Patent Organism Depositary, National Institute of Advanced Industrial Science and Technology on Jun. 27, 2001 in accordance with the Budapest Treaty, and the accession number FERM BP-7646 was allotted thereto.

The publicly known bacterial strain which produces the carbamoylase may specifically include Pseudomonas sp. AJ 11220 strain (JP-P-S56-003034 B). As a result of reidentification, it has been found that Pseudomonas sp. AJ 11220 strain belongs to Agrobacterium sp. Agrobacterium sp. AJ 11220 strain was deposited to Ministry of International Trade and Industry, Agency of Industrial Science and Technology, National Institute of Bioscience and Human-Technology on Dec. 20, 1977, and the accession number FERM-P4347 was allotted thereto. Then, the strain was transferred to International Patent Organism Depositary, National Institute of Advanced Industrial Science and Technology on Jun. 27, 2001 in accordance with the Budapest Treaty, and the accession number FERM BP-7645 was allotted thereto (original date of deposit under the Budapest Treaty: May 1, 1981). It has been known that the L-carbamoylase is present in Bacillus sp. AJ 12299 strain (JP-P-S63-24894 A). Bacillus sp. AJ 12299 strain was deposited to Ministry of International Trade and Industry, Agency of Industrial Science and Technology, National Institute of Bioscience and Human-Technology on Jul. 5, 1986, and the accession number FERM-P8837 was allotted thereto. Then, it was transferred to International Patent Organism Depositary, National Institute of Advanced Industrial Science and Technology on Jun. 27, 2001 in accordance with the Budapest Treaty, and the accession number FERM BP-7646 was allotted thereto.

Cultivation of the cells having the hydantoinase gene, the carbamoylase gene and, if necessary, the hydantoin racemase gene results in expression and production of the hydantoinase, the carbamoylase and, if the DNA encoding the hydantoin racemase gene has been introduced, the hydantoin racemase. The medium for such a production may include an ordinary medium for culturing Escherichia coli, such as an M9-casamino acids medium and an LB medium. More specific conditions for cultivation and induction of the products are appropriately selected depending on types of the marker of the vector and the host bacterium for use.

Cultured cells may be collected by centrifugation. Subsequently, the cells may be disrupted or lysed to collect an enzyme. The enzyme may be used as a crude enzyme solution. The disruption may be performed by ultrasonic disruption, French press disruption and glass bead disruption. The cell lysis may be performed by a treatment with egg white lysozyme, a peptidase treatment and an appropriate combination thereof. If necessary, these enzymes may further be purified for use by the techniques such as an ordinary precipitation, filtration and column chromatography. In this case, a purification method by taking advantage of an antibody against the enzyme may be utilized.

In a preferable embodiment of the protein production on a large scale using the recombinant DNA technology, an association of the protein may occur in the transformant for producing the protein, to constitute an inclusion body of the protein. The advantages of the production method in this manner include the protection of the desired protein from digestion by proteases present in the cells, and ready purification of the desired protein performed by disruption of the cells following centrifugation.

The protein inclusion body obtained in this way may be solubilized by a protein denaturing agent, which may then be subjected to steps for activation and regeneration including removal of the denaturing agent, to be converted into a correctly folded and physiologically active protein. There are many examples of such a procedure, such as activity regeneration of human interleukin 2 (see JP-P-S62-205790 A).

In order to obtain the active protein from the protein inclusion body, a series of the manipulations such as solubilization and activity regeneration is required, and thus the manipulations are more complicated than direct production of the active protein. However, when a large amount of protein produced in the cells affects cell growth, accumulation thereof as the inactive inclusion body in the cells may advantageously suppress the effect caused by such a protein.

Examples of the methods for producing the desired protein on a large scale as the inclusion body may include a method of expressing the protein alone under control of a strong promoter, as well as a method of expressing the desired protein as a fused protein with a protein known to be expressed in a large amount.

It is useful to arrange a recognition sequence of restriction protease at an appropriate position, for cleaving out the objective protein after the expression of the fused protein.

When the protein inclusion body is formed, the protein inclusion body may be collected as the fused protein before solubilizing the protein with the denaturing agent. Although the fused protein may be solubilized together with a cell protein, it is preferable to retrieve the inclusion body before solubilization for facilitating the subsequent purification steps. The inclusion body may be separated from the cells by known methods. For example, the microbial cells are disrupted and the inclusion body is then collected by centrifugation. Examples of the denaturing agent for solubilizing the protein inclusion body may include guanidine hydrochloride (e.g., 6 M, pH 5 to 8) and urea (e.g., 8 M).

The active protein may be regenerated by removing the denaturing agent by dialysis. Examples of a dialysis solution used for the dialysis may include a Tris-hydrochloride buffer and a phosphate buffer. A concentration thereof may be 20 mM to 0.5 M, and a pH value may be 5 to 8.

It is preferable to lower the protein concentration of the regeneration process to about 500 μg/mL or less. In order to prevent the regenerated enzyme protein from self-crosslinking, the dialysis temperature may preferably be regulated at 5° C. or below. Examples of the method for removing the denaturing agent other than the dialysis method may include a dilution method and an ultrafiltration method, and the regeneration of the activity may be expected using any of these methods.

### EXAMPLES

The present invention will be illustrated in more detail with reference to the following non-limiting Examples.

### Example 1

Preparation of E. coli co-expressing an hydantoin racemase gene derived from AJ 3912 strain, and D-hydantoinase gene derived from AJ 11199 strain, and production of N-carbamoyl-D-tyrosine

1-1. Construction of a Plasmid Carrying the Hydantoin Racemase Gene

A promoter region of a trp operon on a chromosomal DNA of Escherichia coli (E. coli) W3110 strain was amplified using the oligonucleotides shown in Table 1 as primers (combination of (1) and (2) in Table 1), and a resulting DNA fragment was ligated to a pGEM-Teasy vector (supplied from Promega). E. coli JM109 strain was transformed with a solution containing this ligation product, and strains having the plasmid in which a trp promoter had been inserted in an opposite direction to that of the lacZ gene were selected among ampicillin resistant strains.

Subsequently, a DNA fragment containing the trp promoter obtained by treating this plasmid with EcoO1091 and EcoRI was ligated to pUC 19 (supplied from Takara) that had been treated with EcoO109I and EcoRI. E. coli JM109 strain was transformed with a solution containing this ligation product, and a strain having the desired plasmid was selected among ampicillin resistant strains. The plasmid was designated pTrp1.

Subsequently, pKK223-3 (supplied from Amersham Pharmacia) was treated with HindIII/HincII, and the obtained DNA fragment containing a rrnB terminator was ligated to pTrp1 that had been treated with HindIII/PvuII. E. coli JM109 strain was transformed with a solution containing this ligation product, and a strain having the objective plasmid was selected from among the ampicillin resistant strains. The plasmid was designated pTrp2.

Subsequently, the trp promoter region thereof was amplified by PCR with pTrp2 as a template and the oligonucleotides shown in Table 1 as the primers (combination of (1) and (3) in Table 1). This DNA fragment was treated with EcoO109I/NdeI, and ligated to pTrp2 that had been treated with EcoO109I/NdeI. E. coli JM109 strain was transformed with a solution containing this ligation product, and a strain having the desired plasmid was selected among ampicillin resistant strains. The plasmid was designated pTrp4.

A 2.4 kb DNA fragment obtained by treating pSTV28 (supplied from Takara) with EcoO109I/PvuI, a 0.9 kb DNA fragment obtained by treating pKK223-3 (supplied from Amersham Pharmacia) with HindIII/PvuI, and a 0.3 kb DNA fragment obtained by treating pTrp4 with EcoO109F/HindIII were ligated. E. coli JM109 strain was transformed with a solution containing this ligation product, and a strain having the desired plasmid was selected from among chloramphenicol resistant strains. The plasmid was designated pTrp8.

TABLE 1
Primers for amplification of trp promoter region

| | | |
|---|---|---|
| (1) 5′ side | GTATCACGAGGCCCTAGCTGTGGTGTCATGGTCGGTGATC | SEQ. ID No.7 |
| | EcoO109I | |
| (2) 3′ side | TTCGGGGATTCCATATGATACCCTTTTTACGTGAACTTGC | SEQ. ID No. 8 |
| | NdeI | |
| (3) 3′ side | GGGGGGGGCATATGCGACCTCCTTATTACGTGAACTTG | SEQ. ID No. 9 |
| | NdeI | |

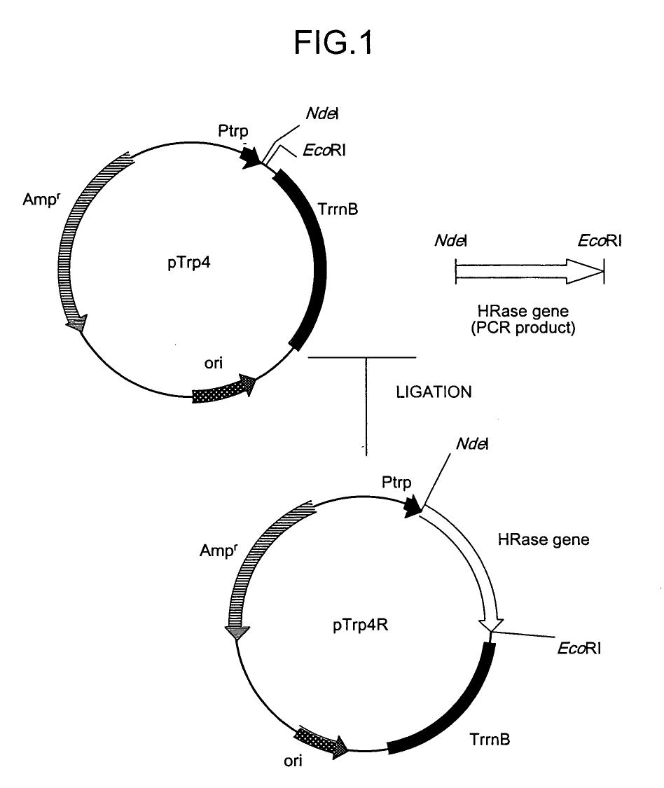

The objective gene was amplified by PCR using a chromosomal DNA of Microbacterium liquefaciens AJ 3912 strain as the template and oligonucleotides shown in Table 2 as the primers. This fragment was treated with NdeI/EcoRI, and the resulting DNA fragment was ligated to a pTrp4 that had been treated with NdeI/EcoRI. E. coli JM109 strain was transformed with a solution containing this ligation product, and a strain having the objective plasmid was selected from among the ampicillin resistant strains. The plasmid was designated pTrP4R (FIG. 1). In the drawings, the trp promoter, the rmB terminator, an ampicillin resistant gene, and the hydantoin racemase gene are represented by “Ptrp”, “TrrnB”, “AMPr”, and “HRase gene”, respectively.

TABLE 2

| | | |
|---|---|---|
| (1) 5′ end | CGGGAATTCCATATGCGTATCCATGTCATCAA | SEQ. ID No. 23 |
| | NdeI | |
| (2) 3′ end | CGCGGATCCTTAGAGGTACTGCTTCTCGG | SEQ. ID No. 24 |
| | EcoRI | |

1-2. Construction of Plasmid Carrying Hydantoin Racemase Gene and D-Hydantoinase Gene

The objective D-hydantoinase gene was amplified by PCR using a chromosomal DNA of Flavobacterium sp. AJ 11 199 strain as the template and oligonucleotides shown in Table 3 as the primers. This fragment was treated with NdeI/EcoRI, and the resulting DNA fragment was designated H-NE.

TABLE 3

Primers for amplification of D-hydantoinase gene derived
from AJ11199

| | | | |
|---|---|---|---|
| (1) | 5′ end | GGGAATTCCATATGACCCATTACGATCTCGTCATTC | SEQ. ID No. 19 |
| | | NdeI | |
| (2) | 3′ end | CGGAATTCTCAGGCCGTTTCCACTTCGCC | SEQ. ID No. 20 |
| | | EcoRI | |

The hydantoin racemase gene containing the objective SD sequence was amplified by PCR with the plasmid pTrp4R obtained in the above step 1-1 carrying the hydantoin racemase gene derived from the AJ 3912 strain as the template, and oligonucleotides shown in Table 4 as the primers. The resulting DNA fragment was ligated to a pGEM-Teasy vector (supplied from Promega). E. coli JM109 strain was transformed with a solution containing this ligation product, and a strain having the plasmid in which the hydantoin racemase gene had been inserted in an opposite direction to that of the lacZ gene was selected from among ampicillin resistant strains. The resulting plasmid was treated with EcoRI/BamHI, and the DNA fragment containing the hydantoin racemase gene was designated R-EB.

TABLE 4

Primers for amplification of hydantoin racemase
gene derived from AJ3912

| | | |
|---|---|---|
| (1) 5′ end | CAAGTTCACGTAATAAGGAGGTCGCATATG | SEQ. |
| | | ID No. |
| | | 21 |
| (2) 3′ end | CGCGGATCCTTAGAGGTACTGCTTCTCGG | SEQ. |
| | BamHI | ID No. |
| | | 22 |

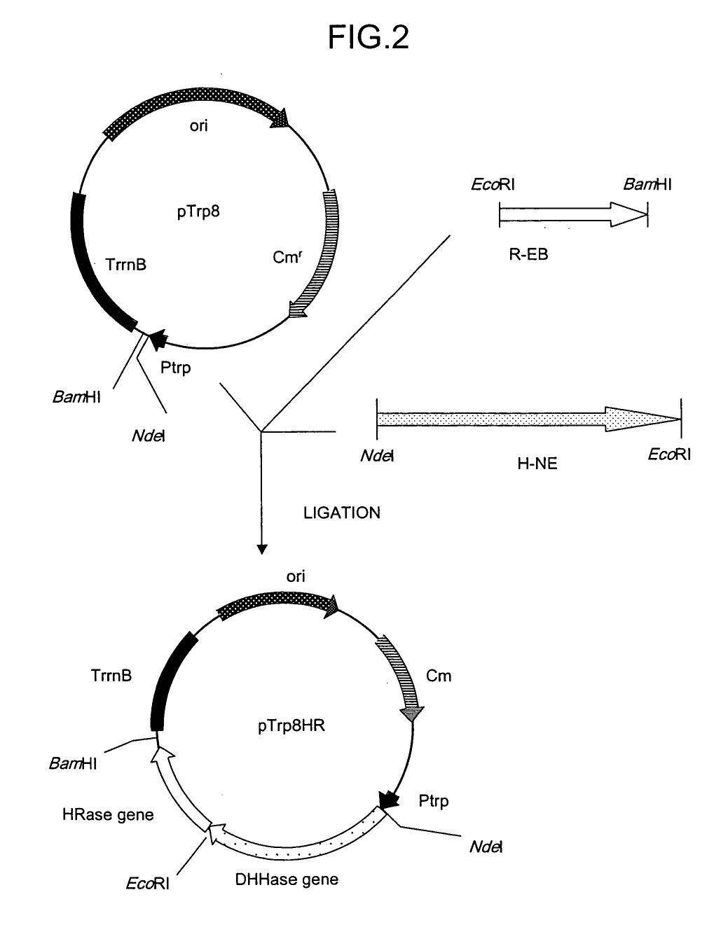

The plasmid pTrp8 treated with NdeI/BamHI was ligated to H—Ne and R-EB. E. coli JM 109 strain was transformed with a solution containing this ligation product, and a strain having the desired plasmid was selected from among chloramphenicol resistant strains. The plasmid was designated pTrp8HR (FIG. 2).

1-3. Production of N-Carbamoyl-D-Tyrosine

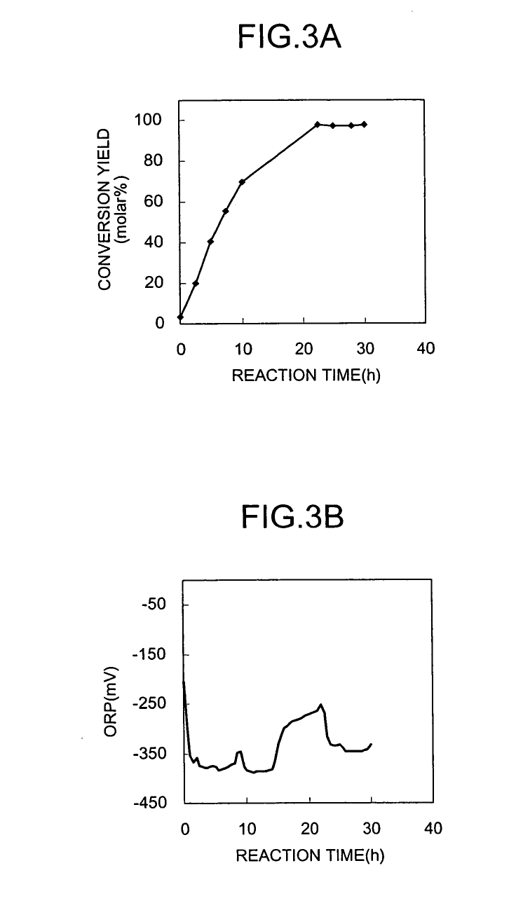

E. coli JM109 strain having pTrp8HR was cultured in an LB medium (50 mL) containing 50 μg/mL of chloramphenicol at 30° C. for 16 hours. Subsequently, 1 mL of the resulting medium was transferred into 300 mL of a medium I (2.5% glucose, 0.5% ammonium sulfate, 0.14% potassium dihydrogen phosphate, 0.23% trisodium citrate dihydrate, 0.002% iron (II) sulfate heptahydrate, 0.1% magnesium sulfate heptahydrate, 0.002% manganese sulfate pentahydrate, 0.0001% thiamine hydrochloride, pH 7.0) containing 50 μg/mL of chloramphenicol, and cultured in a Jar fermenter at 33° C. for 24 hours with controlling pH at 7.0 and a dissolved oxygen concentration at 1.5 ppm or more. Subsequently, 15 mL of the resulting medium was transferred into a medium II (2.5% glucose, 0.5% ammonium sulfate, 0.3% phosphoric acid, 0.23% trisodium citrate dihydrate, 0.1% magnesium sulfate heptahydrate, 0.002% iron (II) sulfate heptahydrate, 0.002% manganese sulfate pentahydrate, 0.0001% thiamine hydrochloride, pH 7.0) containing 50 μg/mL of chloramphenicol, and cultured in a Jar fermenter at 35° C. for 24 hours while controlling pH at 7.0 and the dissolved oxygen concentration at 1.5 ppm or more, and adding an aqueous solution of 50% glucose at 3.5 mL/hr 9 hours after the onset of the cultivation. Then, 225 mL of the resulting medium (dried microbial cell weight: about 9 g) was added to 4.275 L of a substrate solution (2.6 g/dL DL-5-(4-hydroxybenzyl)hydantoin, 1.05 mM magnesium sulfate, 21 mM KPB, pH 7.5), and a reaction was performed at 37° C. under a nitrogen atmosphere while keeping a reaction solution at pH 9.0 (adjusted with 8 N NaOH) and bubbling the reaction solution with nitrogen gas. During the reaction, an oxidation-reduction potential of the reaction solution was monitored using an oxidation-reduction electrometer (trade name: F-935-B120-DH, supplied from Broadly James). Time-course changes thereof were as shown in FIG. 3B. Throughout the reaction, the indicated value of a dissolved oxygen meter which measured a dissolved oxygen amount in the aqueous solution was 0.3 ppm or less. The amount of N-carbamoyl-D-tyrosine produced in the reaction solution was measured several times after the onset of the reaction to calculate a conversion yield (mol %), which was changed as shown in FIG. 3A. A yield of 2.9 g/dL of N-carbamoyl-D-tyrosine (molar yield: 97%) after 30 hours of the reaction was observed.

### Example 2

Preparation of E. coli co-expressing hydantoin racemase gene derived from AJ 3912 strain, D-hydantoinase gene, and D-carbamoylase gene derived from AJ 11199 strain, and production of D-tyrosine

2-1. Construction of Plasmid Carrying the D-Hydantoinase Gene and D-Carbamoylase Gene

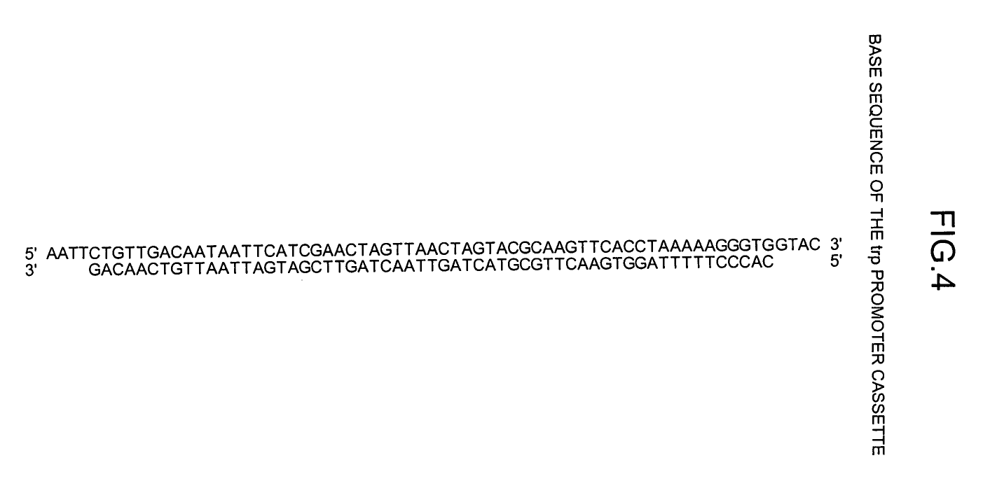

A plasmid pHSG298 (supplied from Takara) was treated with EcoRI/KpnI, the resulting DNA fragment was ligated to a trp promoter cassette (FIG. 4), and E. coli JM109 strain was transformed with a solution containing this ligation product. A strain having the desired plasmid was selected from among the kanamycin resistant strains, and the desired plasmid was designated pTrp298EK. In SEQ ID NO:10, a sequence of an upper chain of the trp promoter cassette shown in FIG. 4 was described.

The objective D-hydantoinase gene was amplified by PCR using a chromosomal DNA of Flavobacterium sp. AJ 11199 strain as the template and oligonucleotides shown in Table 5 as the primers. This fragment was treated with KpnI/XbaI, and the resulting DNA fragment was ligated to pTrp298EK that had been treated with KpnI/XbaI. E. coli JM109 strain was transformed with a solution containing this ligation product, and a strain having the desired plasmid was selected among kanamycin resistant strains. The plasmid was designated pTrp298DHHase3.

TABLE 5

Primers for amplification of D-hydantoinase
gene derived from AJ 11199

| | | |
|---|---|---|
| (1) 5′ end | GGGGTACCATGACCCATTACGATCTC | SEQ. ID No. |
| | KpnI | 11 |
| (2) 3′ end | GCTCTAGACGTCCTGTCCTTTCCGCC | SEQ. ID No. |
| | XbaI | 12 |

The objective D-carbamoylase gene was amplified by PCR using a chromosomal DNA of Flavobacterium sp. AJ 11199 strain as the template and oligonucleotides shown in Table 6 as the primers. This fragment was treated with KpnI/XbaI, and the resulting DNA fragment was ligated to pTrp298EK that had been treated with KpnI/XbaI. E. coli JM109 strain was transformed with a solution containing this ligation product, and a strain having the desired plasmid was selected from among the kanamycin resistant strains. The plasmid was designated pTrp298DCHase1.

Subsequently, the plasmid pTrp298DCHase1 was treated with EcoRI/XbaI to yield a DNA fragment in which the trp promoter was linked to the D-carbamoylase gene. This DNA fragment was ligated to pHSG299 (supplied from Takara) that had been treated with EcoRI/XbaI. E. coli JM109 strain was transformed with a solution containing this ligation product, and a strain having the desired plasmid was selected from among the kanamycin resistant strains. The plasmid was designated pTrp299DCHase1.

TABLE 6

Primers for amplification of D-carbamoylase
gene derived from AJI1199

| | | |
|---|---|---|
| (1) 5′ end | GGGGTACCGGAGAGGAATATGCCAGG | SEQ. ID No. |
| | KpnI | 13 |
| (2) 3′ end | GCTCTAGAGCCGCGCCGCTCAGACGG | SEQ. ID No. |
| | XbaI | 14 |

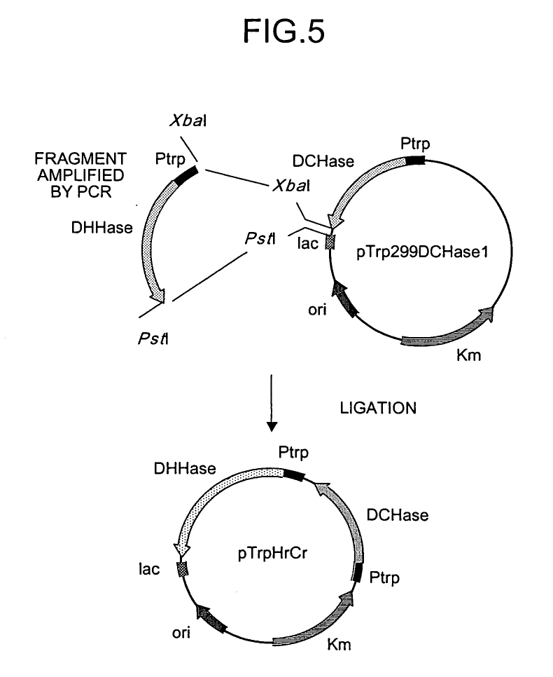

PCR was performed using pTrp298DHHase3 as the template and the oligonucleotides shown in Table 7 as the primers. The resulting DNA fragment was treated with XbaI/PstI, and then ligated to pTrp299DCHase1 that had been treated with XbaI/PstI. E. coli JM109 strain was transformed with a solution containing this ligation product, a strain having the desired plasmid was selected from among the kanamycin resistant strains, and the plasmid was designated pTrpHrCr (FIG. 5). In the drawings, the D-hydantoinase gene is represented by “DHHase” or “DHHase gene”, the D-carbamoylase gene is represented by “DCHase” or “DCHase gene”, and a kanamycin resistant gene is represented by “Km”.

TABLE 7

Primers for amplification of D-hydantoinase gene
and D-carbamoylase gene

| | | |
|---|---|---|
| (1) 5′ end | CGTCTAGATGTTGACAATTAATCAT | SEQ. |
| | XbaI | ID No. |
| | | 15 |
| (2) 3′ end | CGCTGCAGTCAGGCCGTTTCCACTTCGCCCGT | SEQ. |
| | PstI | ID No. |
| | | 16 |

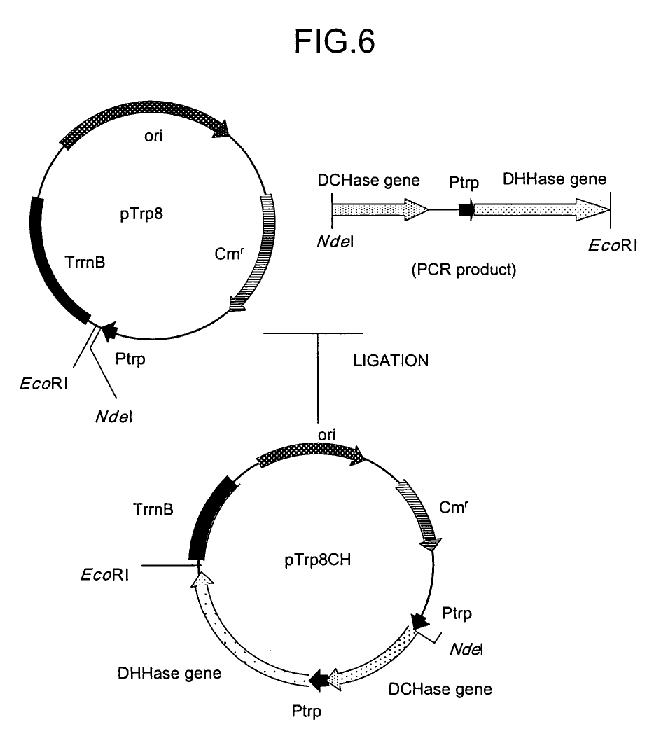

The desired gene was amplified by PCR using pTrpHrCr as the template and the oligonucleotides shown in Table 8 as the primers. This fragment was treated with NdeI/EcoRI, and the resulting DNA fragment was ligated to pTrp8 that had been treated with NdeI/EcoRI. E. coli JM109 strain was transformed with a solution containing this ligation product, and a strain having the desired plasmid was selected from among the chloramphenicol resistant strains. The plasmid was designated pTrp8CH (FIG. 6). In the drawings, a chloramphenicol resistant gene is represented by “Cm^{r}”. Table 8. Primers for amplification of D-hydantoinase gene and D-carbamoylase gene

TABLE 8

Primers for amplification of D-hydantoinase gene
and D-carbamoylase gene

| | | |
|---|---|---|
| (1) 5′ end | GGAATTCCATATGCCAGGAAAGATCATTCTCGCG | SEQ. |
| | NdeI | ID No. |
| | | 17 |
| (2) 3′ end | CGGAATTCTCAGGCCGTTTCCACTTCGCC | SEQ. |
| | EcoRI | ID No. |
| | | 18 |

2-2. Production of D-Tyrosine

E. coli JM109 strain having two plasmids, pTrp4R prepared in the step 1-1 in Example 1 and pTrp8CH prepared in the aforementioned step, were cultured in the LB medium (50 mL) at 30° C. for 16 hours. Subsequently, 1 mL of the resulting medium was transferred into 300 mL of the medium I (2.5% glucose, 0.5% ammonium sulfate, 0.14% potassium dihydrogen phosphate, 0.23% trisodium citrate dihydrate, 0.002% iron (II) sulfate heptahydrate, 0.1% magnesium sulfate heptahydrate, 0.002% manganese sulfate pentahydrate, 0.0001% thiamine hydrochloride, pH 7.0), and cultured in a Jar fermenter at 33° C. for 24 hours while controlling pH at 7.0 and a dissolved oxygen concentration at 1.5 ppm or more. Subsequently, 15 mL of the resulting medium was transferred into 300 mL of the medium II (2.5% glucose, 0.5% ammonium sulfate, 0.3% phosphoric acid, 0.23% trisodium citrate dihydrate, 0.1% magnesium sulfate heptahydrate, 0.002% iron (II) sulfate heptahydrate, 0.002% manganese sulfate pentahydrate, 0.0001% thiamine hydrochloride, pH 7.0), and cultured in a Jar fermenter at 35° C. for 24 hours while controlling pH at 7.0 and the dissolved oxygen concentration at 1.5 ppm or more, and adding an aqueous solution of 50% glucose at 3.5 mL/hr 9 hours after the onset of the cultivation.

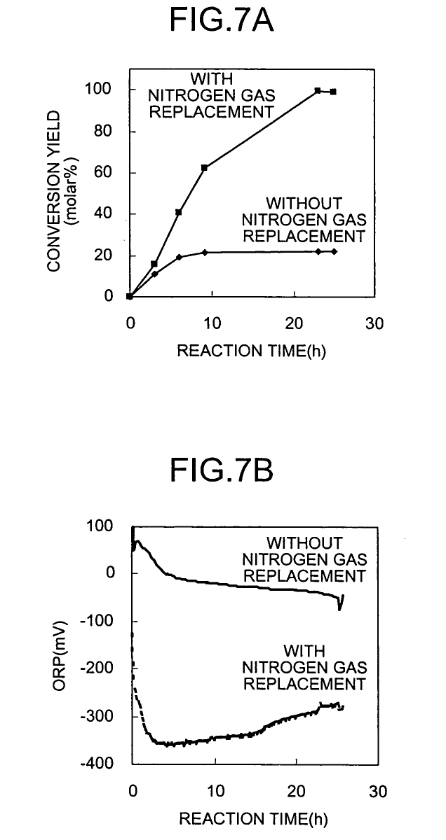

Then, 15 mL of the resulting medium (dried microbial cell weight: about 600 mg) was added to 285 mL of a substrate solution (3.1 g/dL DL-5-(4-hydroxybenzyl)hydantoin, 1.05 mM magnesium sulfate, 21 mM KPB, pH 7.5), and a reaction was performed at 37° C. under a nitrogen atmosphere while keeping a reaction solution at pH 7.5 (adjusted with 1 N NaOH and 2 N H₂SO₄) and bubbling the reaction solution with a nitrogen gas. During the reaction, an oxidation-reduction potential (ORP) of the reaction solution was monitored using the oxidation-reduction electrometer (trade name: F-935-B120-DH, supplied from Broadly James). The time-course changes were as shown by a curve “with nitrogen gas replacement” in FIG. 7B. Throughout the reaction, the indicated value of the dissolved oxygen meter which measured the dissolved oxygen amount in the aqueous solution was 0.3 ppm or less. The amount of D-tyrosine produced in the reaction solution was measured several times after the onset of the reaction to calculate a conversion yield (mol %), which was changed as shown by a curve “with nitrogen gas replacement” in FIG. 7A. A yield of 3.0 g/dL of D-tyrosine (molar yield: 99%) after 24 hours of the reaction was observed.

### Comparative Example 1

D-tyrosine was produced in the same way as in Example 1, except that the reaction of the medium with the substrate solution was carried out in ambient air without bubbling the reaction solution with nitrogen gas. The time-course changes of the oxidation-reduction potential in the reaction solution are shown by a curve with “without nitrogen gas replacement” in FIG. 7B. The conversion yield of D-tyrosine was changed as shown by a curve with “without nitrogen gas replacement” in FIG. 7A. A yeild of 0.6 g/dL of D-Tyr (molar yield: 22%) after 24 hours of the reaction was observed.

### INDUSTRIAL APPLICABILITY

As explained in the above, the method for producing the optically active amino acid according to the present invention is useful for the efficient production of the optically active amino acid, and particularly suitable for the industrial production of the optically active amino acids such as D-isomers or L-isomers of tyrosine, tryptophan, and o-benzylserine.

Although the present invention has been described with reference to the preferred examples, it should be understood that various modifications and variations can be easily made by those skilled in the art without departing from the spirit of the invention. Accordingly, the foregoing disclosure should be interpreted as illustrative only and is not to be interpreted in a limiting sense. The present invention is limited only by the scope of the following claims along with their full scope of equivalents. All documents cited herein including Japanese Patent Application No.2004-095983, are hereby incorporated by reference

1. A method for producing an optically active amino acid comprising contacting a 5-substituted hydantoin with a group of enzymes comprising hydantoinase and carbamoylase in an aqueous solution with a dissolved oxygen concentration of 1.5 ppm or less.

2. The method according to claim 1 wherein said group of enzymes further comprises a hydantoin racemase.

3. The method according to claim 1 wherein said hydantoinase and said carbamoylase are derived from cells having a hydantoinase gene and a carbamoylase gene.

4. The method according to claim 2 wherein said hydantoin racemase is derived from cells having a hydantoin racemase gene.

5. The method according to claim 1 wherein said aqueous solution is placed under an atmosphere replaced with an inert gas.

6. The method according to claim 3 wherein said cells comprise Escherichia coli.

7. The method according to claim 1 wherein said optically active amino acid comprises tyrosine.

8. The method according to claim 7 wherein said tyrosine comprises D-tyrosine.
